(19) United States
(12) Patent Application Publication
(10) Pub. No.: US 2007/0260189 A1
(43) Pub. Date: Nov. 8, 2007

(54) Syringe with Recessed Nose for Use with Frontal Attachments

(76) Inventors: Thomas J. Shaw, Frisco, TX (US); Ni Zhu, Plano, TX (US)

Correspondence Address: Monfy L. Ross, Esq.;LOCKE LIDDELL & SAPP LLP, Suite 2200, 2200 Ross Avenue, Dallas, TX, 75201-6776, US

(21) Appl. No.: 11/743,706

(22) Filed: May 3, 2007

Related U.S. Application Data
(60) Provisional application No. 60/798,433, filed on May 5, 2006.

(51) Int. Cl. A61M 5/00

(52) U.S. Cl. 604/187

(57) ABSTRACT

A syringe for use with frontal attachments with a forwardly extending projecting structure, which recesses the nose of the syringe and keeps the recessed nose from becoming contaminated by physical contact. In one preferred embodiment, the projecting structure is tubular and internally threaded such that a standard CLAVE® connector can be attached to a pre-filled syringe at such time as the syringe is to be used. In another preferred embodiment, the projecting structure is tubular and internally threaded such that a luer lock needle attachment can be attached to a luer lock syringe at such time as the syringe is to be used.

### RELATED APPLICATION

This application claims the benefit of U.S. Provisional patent application Ser. No. 60/798,433 filed May 5, 2006 and entitled “Syringe with Recessed Nose for Use with Frontal Attachments.”

### BACKGROUND OF THE INVENTION

1. Field of the Invention

This invention relates to a medical device and more particularly to a syringe, either traditional or pre-filled, having a recessed nose section adapted to prevent contamination from human contact when connecting a frontal attachment such as a CLAVE® connector for introducing medication into a closed IV system or a luer lock needle attachment.

2. Description of Related Art

Syringes which use frontal attachments rather than having an affixed or preset retractable needle are known in the prior art. Various frontal attachments are used with these syringes, and generally, the frontal attachments are threaded onto a piece that encircles the nose of the syringe. The nose of the syringes of the prior art extends beyond this encircling structure and comprises the leading edge of the syringe. There are two main types of syringes which use frontal attachments: pre-filled syringes and standard syringes which are shipped without an affixed needle.

Certain types of pre-filled syringes are adapted for use with various attachments such as the CLAVE brand connector marketed by ICU Medical and disclosed in U.S. Pat. No. 5,694,686. With this design, when the syringe is ready to use, an attachment is connected to the front. This attachment has a rearwardly facing needle or other sharp object which punctures the sealing membrane from the outside. The syringe plunger is depressed and the pre-filled liquid flows out of the syringe through the shaft of the puncturing needle, through the attachment and into a closed IV system. Pre-filled syringes that have a removable sealing cap rather than a sealing membrane, operate with attachments in a similar manner. The cap is removed and the attachment is connected to the nose portion of the syringe via the internal threads on the encircling structure. The syringe plunger is depressed and pre-filled liquid flows through the attachment.

Certain syringes which do not have a permanently affixed needle use the luer lock mechanism to attach or remove the needle. In this type of syringe, the needle is held by a luer lock needle attachment having either a flange or external threads which is capable of being threaded onto the syringe. Similarly to the pre-filled syringe discussed above, the luer lock syringe has a threaded, nose-encircling structure which is adapted to accept the flange or threads of the luer lock needle attachment.

The main disadvantage with either of these types of syringes is that the nose portion of the syringe body is exposed to contamination due to its position as the exposed front edge of the syringe. The nose can become easily contaminated through human contact. The rise of potentially deadly staph and other infections as a result of the use of pre-filled and/or standard syringes in conjunction with needleless systems, and accessing ports that are integrated within needleless systems, is solved by the present invention by recessing the nose of the syringe behind an extended projection or projections, that in the preferred embodiment is an encircling outer wall. By extending the projections beyond the tip of the nose, the nose is no longer the front edge of the syringe and is protected from contamination due to human contact.

### SUMMARY OF THE INVENTION

Syringes for use with frontal attachments are disclosed herein that provide significant advantages over the prior art. The syringes of the present invention are designed such that the nose of the syringes are recessed behind a projection or projections that extend beyond the tip of the nose. In the preferred embodiment the projections are an encircling outer wall, which provides the means by which the frontal attachments are connected to the syringe. By extending the outer wall beyond the nose, the recessed nose is protected from contamination due to accidental contact with contaminants prior to affixing the frontal attachment.

The first embodiment disclosed is that of a pre-filled syringe for use with a CLAVE® connector that provides significant advantages over the pre-filled syringes of the prior art. The pre-filled syringe of this invention is designed such that the nose of the syringe is recessed inside of a surrounding outer wall that extends beyond the tip of the nose. The recessed nose is protected from contamination due to accidental contact with contaminants prior to attachment of the CLAVE® connector. The nose is not exposed to human contact, which helps prevent the spread of disease.

The pre-filled syringe of the invention comprises an elongated hollow syringe body made from either glass or plastic. The syringe body has an open nose with a reduced cross-sectional area in the front and an open back end. Disposed in the hollow body is a plunger comprising a plunger seal in sliding sealed contact with the interior wall of the body. The one-piece plunger seals the back end of the syringe body and has a surface for applying thumb force during injections. The nose of the syringe body is sealed with a removable sealing cap. Because it is sealed on both ends, the syringe can be pre-filled with liquid and stored until it is used.

The nose of the disclosed pre-filled syringe is protected by and recessed behind a forwardly projecting structure, which is part of the syringe body. This forwardly projecting structure prevents contaminating physical contact between the user and the recessed nose after the sealing cap has been removed. The forwardly projecting structure does not completely enclose and cover the recessed nose; it simply protrudes past the tip to prevent human contact with the nose.

In a preferred embodiment of the invention, the projecting structure recessing the nose of the syringe body is configured so that a CLAVE® connector can be attached at the time of use. A portion of this projecting structure encircles the nose and is threaded to accept the external threads from a CLAVE® connector. The portion of this projecting structure that projects beyond the nose can either be a solid, encircling wall or another equivalent structure, such as a plurality of fingers, that do not solidly encircle the nose. The projecting structure, whether solidly encircling or not, must block, interfere with or otherwise prevent human physical contact with the nose to protect the nose from contamination.

At the time of use, the removable sealing cap is removed as the pre-filled syringe is being held upright, and the CLAVE® connector is attached. In this embodiment, the projecting structure encircles the nose of the syringe body and extends a distance beyond the tip. The inner wall of the encircling projecting structure is cooperatively threaded to accept the corresponding threads of a CLAVE® connector. The CLAVE® connector has a rearwardly facing needle inside protected by an elastomeric support, and the needle is in fluid communication with forward end of the connector. Threading the CLAVE® connector onto the pre-filled syringe causes the elastomeric support to move relative to the static needle and expose the needle. The nose of the syringe and elastomeric support form a fluid tight seal when the CLAVE® connector is fully threaded onto the pre-filled syringe. Then, as the plunger of the pre-filled syringe is depressed, the medication flows through the nose of the syringe, through the needle of the CLAVE® connector and out through the forward end of the connector and into a closed IV system. In a closed IV system, the forward end of a CLAVE® connector is attached to an IV tube, which is in fluid communication with a fluid bottle. The fluid bottle has another IV tube, which is inserted into a patient's vein.

An alternative embodiment is disclosed wherein the nose-recessing projecting structure is incorporated onto a standard, non-pre-filled syringe configured so that a needle can be attached using a luer lock mechanism. Luer lock mechanisms are well known in the art and generally consist of a luer lock needle holder attachment that threads into an internally threaded ring on the front edge of a syringe. This embodiment differs from other luer lock syringes in that the luer lock threaded projecting structure is extended beyond the tip of the nose; thus recessing the nose and protecting it from contamination due to human contact.

In a preferred configuration of both embodiments, the projecting structure is permanently affixed or attached to the syringe body during manufacture. However, it is also understood that the projecting structure can be a separate piece that the user can attach to the syringe prior to use. The body of the syringe and the separate projecting structure can have cooperating threads to allow the projecting structure to be threaded onto the body, or the separate projecting structure can be attached via friction or snap fit through the use of corresponding recesses and bosses or small retaining lips.

The novel features and construction of the present invention, as well as additional objects thereof, will be understood more fully from the following description when read in connection with the accompanying drawings.

### BRIEF DESCRIPTION OF THE DRAWINGS

The apparatus of the invention is further described and explained in relation to the following figures of the drawings wherein:

FIG. 1 is an elevation view, partially in section, of the pre-filled syringe with the sealing cap in place.

FIG. 2 is an elevation view, partially in section, of the pre-filled syringe with the sealing cap removed.

FIG. 3 is an elevation view, partially in section, of the pre-filled syringe and CLAVE® connector prior to attachment of the CLAVE® connector;

FIG. 4 is an elevation view, partially in section, of the pre-filled syringe and CLAVE® connector after the CLAVE® connector has been attached;

FIG. 5 is a detailed view of the area in oval 5 of FIG. 4 better illustrating the connection between the pre-filled syringe and the CLAVE® connector;

FIG. 6 is an elevation view, partially in section, of the luer lock syringe and luer lock needle prior to attachment of the luer lock needle;

FIG. 7 is an elevation view, partially in section, of the luer lock syringe and luer lock needle after the luer lock needle has been attached;

FIG. 8 is a partially exploded perspective view of the syringe showing the solidly encircling projecting structure; and

FIG. 9 is a partially exploded perspective view of the syringe showing an alternative non-solidly encircling projecting structure.

Like reference numerals are used to describe like parts in all figures of the drawings.

### DESCRIPTION OF THE PREFERRED EMBODIMENTS

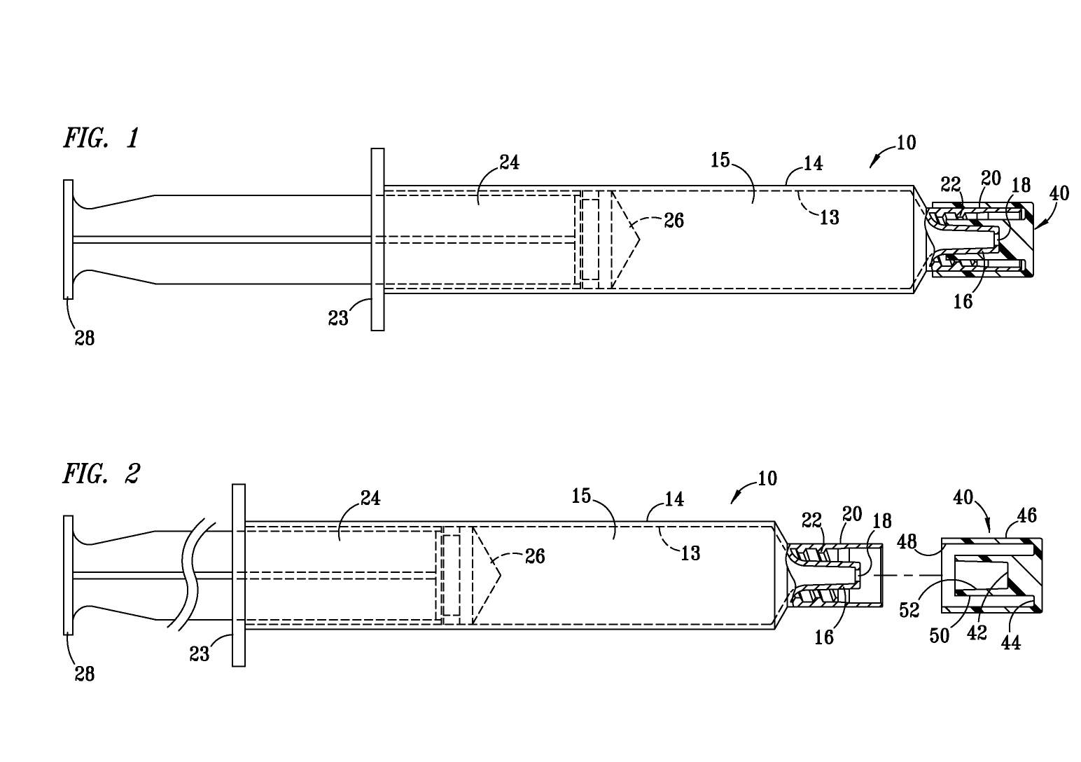

Referring to FIGS. 1 and 2, pre-filled syringe 10 preferably comprises tubular housing 14 and plunger 24. Housing 14 comprises a front end portion or nose 16, which has a reduced cross-sectional area relative to the housing 14, and an open back end portion 23. Housing 14 is preferably molded from a substantially transparent polymeric resin such as polypropylene, but other polymeric resins or glass are also acceptable materials.

Disposed in the back end portion 23 of the housing 14 is a plunger 24 having a plunger seal 26, which may or may not be a separate piece, in sliding sealed contact with interior wall 13 of housing 14. One-piece plunger 24 seals back end portion 23 of syringe housing 14 and has an end cap 28 for applying thumb force to depress plunger 24 and discharge medication from syringe 10 during use. Nose 16 of syringe 10 has opening 18, and nose 16 is sealed prior to use with removable sealing cap 40. Because both the open back end portion 23 and nose 16 of the syringe housing 14 are sealed prior to use, syringe 10 can be pre-filled with liquid in variable fluid chamber 15. Once the syringe 10 has been filled, vacuum pressure along with friction between plunger 24 and interior wall 13 will keep plunger 24 in place.

Projecting structure 20 of pre-filled syringe 10 extends forward past nose 16. Sealing cap 40 is a two-walled structure that slidingly fits over both projecting structure 20 and nose 16. Inner edge 48 of outer wall 46 of sealing cap 40 slidingly engages the outside of projecting structure 20. Inner edge 52 of inner wall 50 of sealing cap 40 slidingly engages the outside of nose 16. As sealing cap 40 is slid onto pre-filled syringe 10, projecting structure 20 seats against stop 44 and nose 16 seats against stop 42. By nose 16 seating against stop 42, nose opening 18 is sealed. Alternatively, sealing cap 40 may have an elastomeric plug placed in nose opening 18, in which case outer wall 46 would be unnecessary in order to achieve a fluid-tight seal. At the time of use, sealing cap 40 is removed from pre-filled syringe 10, thus unsealing nose opening 18.

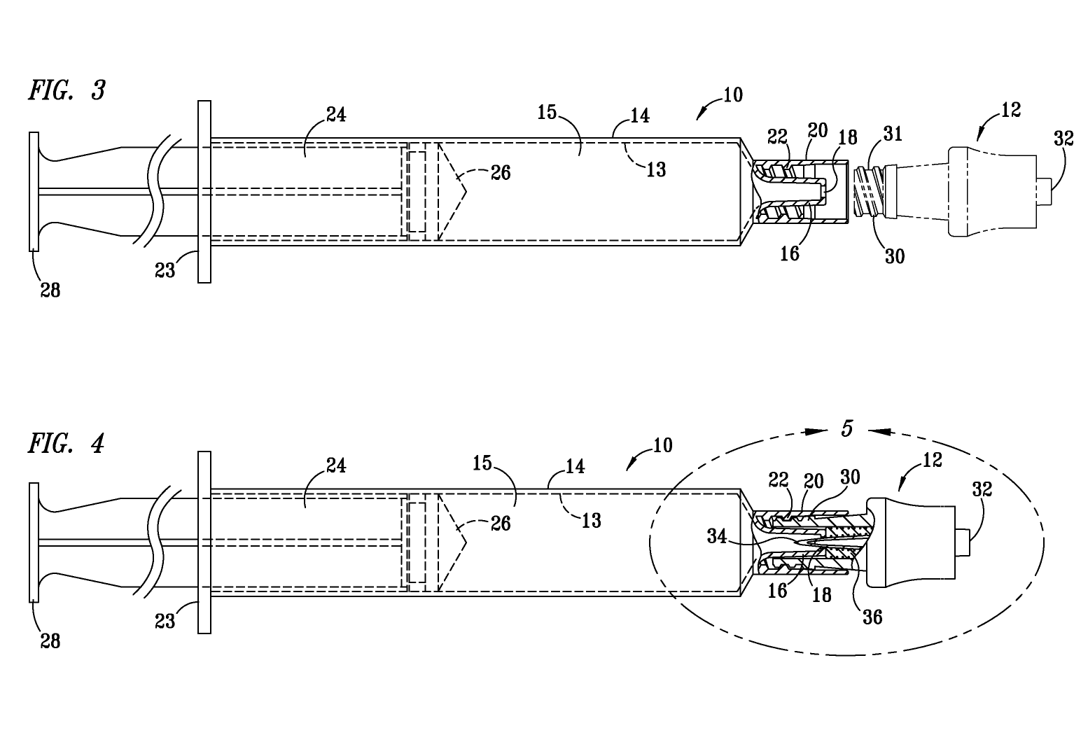

Referring to FIGS. 3 and 4, in a preferred embodiment inner wall 22 of projecting structure 20 is threaded to accept the corresponding threads 30 on a CLAVE® connector 12. The projecting structure 20 is sized so that it extends beyond nose 16 and so that the inner diameter is slightly larger than the outer diameter of CLAVE® connector 12.

A standard CLAVE® connector 12 has a forwardly facing open end 32, which is connectable to a closed IV system. The CLAVE® connector 12 has a back section 31, which is covered with external threads 30. External threads 30 allow CLAVE® connector 12 to be threaded into projecting structure 20 of pre-filled syringe 10. Referring specifically to FIG. 4, CLAVE® connector 12 has a rearwardly facing needle 34 that is encircled by back section 31. Rearwardly facing needle 34 is in fluid communication with open front end 32. External threads 30 are part of an encircling structure that protects and recesses rearwardly facing needle 34 of the CLAVE® connector 12. Elastomeric support structure 36 prevents needle 34 from moving laterally.

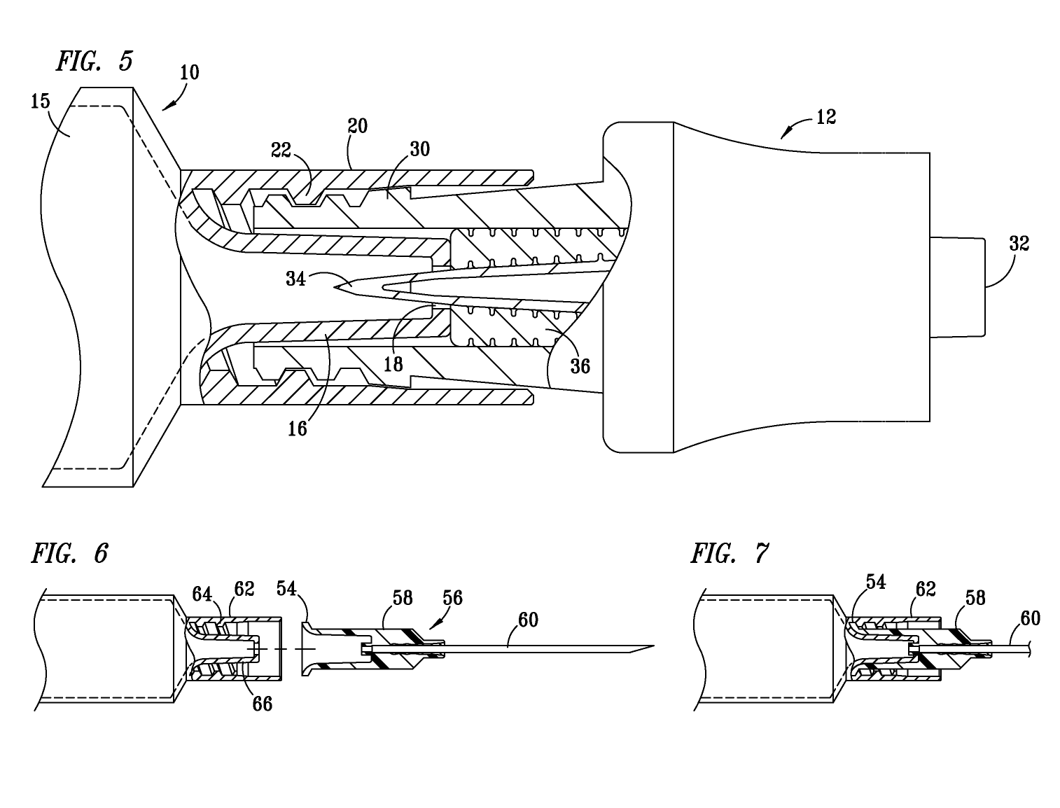

Referring to FIG. 5, pre-filled syringe 10 and CLAVE® connector 12 are threaded together by use of internal threads 22 of projecting structure 20 and external threads 30 of CLAVE® connector 12. As CLAVE® connector 12 is being threaded onto pre-filled syringe 10, elastomeric support structure 36 moves relative to needle 34, thus exposing needle 34. When fully threaded together, elastomeric support structure 36 of CLAVE® connector 12 and nose 16 of pre-filled syringe 10 form a fluid tight seal. By attaching CLAVE® connector 12 to pre-filled syringe 10, rearwardly facing needle 34 of CLAVE® connector 12 protrudes through nose opening 18. Medication flows from variable fluid chamber 15, through nose 16, through needle 34 and out CLAVE® connector 12 though forwardly facing open end 32.

While a preferred embodiment of the invention is shown in FIGS. 1-5, it will be understood by those of ordinary skill in the art upon reading this disclosure that projecting structure 20 of pre-filled syringe 10 can similarly be designed to accept attachments other than a CLAVE® connector 12 provided that the projecting structure 20 extends beyond nose 16 so that nose 16 is recessed and protected from contamination. While a CLAVE® connector attachment is threaded with projecting structure 20, it is foreseeable that an internally smooth projecting structure could be used to protect nose 16 that would be useable for other non-threaded attachments, such as attachments that are held in place relative to the syringe via a friction or press fit.

Referring to FIGS. 6 and 7, another preferred embodiment of the invention is shown wherein projecting structure 62 extends forward past nose 66. Projecting structure 62 has internal threads 64 that are sized to accept flange 54 of luer lock needle attachment 56. Luer lock needle attachment 56 comprises needle 60 and needle holder 58, wherein needle holder 58 has flange 54. Flange 54 of luer lock needle attachment 56 is threaded into projecting structure 62 creating a fluid tight seal between nose 66 and luer lock needle attachment 56. The luer lock embodiment of FIGS. 6 and 7 is intended only for exemplary purposes and it is understood that there are many other ways to temporarily attach a needle to the front of a syringe beyond the threading structure that is shown in which contamination from human contact can be prevented by recessing the nose behind a projecting structure.

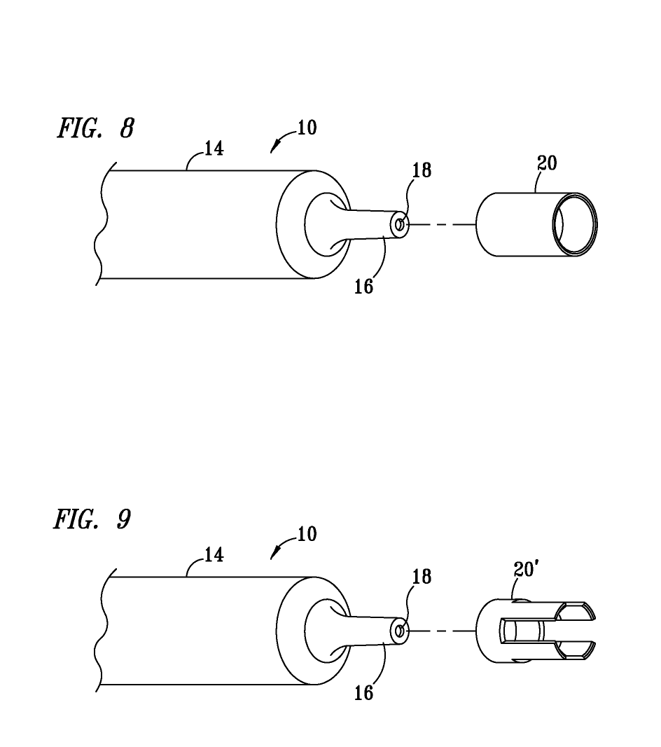

Referring to FIG. 8, in a preferred embodiment, projecting structure 20 is a rigid wall that completely encircles nose 16 of syringe 10. Projecting structure 20 attaches to syringe body 14 and extends beyond nose 16. Referring to FIG. 9, an alternative embodiment is shown wherein projecting structure 20′ is not a solid encircling wall but instead comprises a plurality of protruding fingers, which extend beyond nose 16 of syringe 10.

Other alterations and modifications of the invention will likewise become apparent to those of ordinary skill in the art upon reading the present disclosure, and it is intended that the scope of the invention disclosed herein be limited only by the broadest interpretation of the appended claims to which the inventors are legally entitled.

1. A pre-filled syringe comprising:
an elongated hollow syringe housing having an open nose with a reduced cross-sectional area relative to the housing, a projecting structure extending beyond and recessing the nose, a back end portion with an opening, and a longitudinally extending wall between the nose and the back end portion;
a means for sealing the nose;
an elongated plunger disposed in the back end portion in sliding sealed contact with the longitudinally extending wall of the housing, the plunger having a front end portion and a back end portion; and
a fluid chamber disposed in the housing between the sealed nose and the front end portion of the plunger.

2. The pre-filled syringe of claim 1 wherein the means for sealing the nose is a removable sealing cap removably disposed over the nose.

3. The pre-filled syringe of claim 1 wherein the projecting structure encircles the nose of the syringe housing.

4. The pre-filled syringe of claim 1 wherein the projecting structure is internally threaded and sized to accept external threads from a standard CLAVE® connector.

5. The pre-filled syringe of claim 3 wherein the internally threaded projecting structure completely encircles the nose of the syringe housing.

6. The pre-filled syringe of claim 1 wherein the syringe housing is polymeric.

7. The pre-filled syringe of claim 1 wherein the syringe housing is glass.

8. The pre-filled syringe of claim 1 wherein the projecting structure is separate from and attachable to the housing.

9. The pre-filled syringe of claim 8 wherein the projecting structure is threaded onto the housing.

10. The pre-filled syringe of claim 8 wherein the projecting structure is attached to the housing via a friction fit.

11. A pre-filled syringe comprising:
an elongated hollow syringe housing having an open nose with a reduced cross-sectional area relative to the housing, a tubularly projecting structure extending beyond and recessing the nose, a back end portion with an opening, and a longitudinally extending wall between the nose and the back end portion;
a removable sealing cap disposed over the nose;
an elongated plunger disposed in the back end portion in sliding sealed contact with the longitudinally extending wall of the housing, the plunger having a front end portion with a plunger seal and a back end portion carrying an end cap; and
a fluid chamber disposed in the housing between the removable sealing cap and the front end portion of the plunger.

12. The pre-filled syringe of claim 11 wherein the tubularly projecting structure is internally threaded.

13. The pre-filled syringe of claim 12 wherein the tubularly projecting structure is sized to accept external threads of a standard CLAVE® connector.

14. A syringe comprising:
an elongated hollow syringe housing having an open nose with a reduced cross-sectional area relative to the housing, a projecting structure extending beyond and recessing the nose, a back end portion with an opening, and a longitudinally extending wall between the nose and the back end portion, wherein the projecting structure prevents user contact with the nose;
an elongated plunger disposed in the back end portion in sliding sealed contact with the longitudinally extending wall of the housing, the plunger having a front end portion with a plunger seal and a back end portion; and
a variable fluid chamber disposed in the housing between the nose and the front end portion of the plunger.

15. The syringe of claim 14 wherein the projecting structure encircles the nose of the syringe housing.

16. The syringe of claim 14 wherein the projecting structure is internally threaded and sized to accept a flange from a luer lock needle attachment.

17. The syringe of claim 16 wherein the internally threaded projecting structure completely encircles the nose of the syringe housing.

18. The syringe of claim 14 wherein the projecting structure is separate from and attachable to the housing.

19. The syringe of claim 18 wherein the projecting structure is threaded onto the housing.

20. The pre-filled syringe of claim 18 wherein the projecting structure is attached to the housing via a friction fit.

21. A syringe comprising:
an elongated hollow syringe housing having an open nose with a reduced cross-sectional area relative to the housing, a projecting structure extending beyond and recessing the nose, a back end portion with an opening, and a longitudinally extending wall between the nose and the back end portion;
an elongated plunger disposed in the back end portion in sliding sealed contact with the longitudinally extending wall of the housing, the plunger having a front end portion with a plunger seal and a back end portion carrying an end cap; and
a variable fluid chamber disposed in the housing between the nose and the front end portion of the plunger.

22. The syringe of claim 21 wherein the projecting structure is internally threaded.

23. The syringe of claim 22 wherein the projecting structure is sized to accept a flange from a luer lock needle attachment.

24. A syringe comprising:
an elongated hollow syringe housing having an open nose with a reduced cross-sectional area relative to the housing, a tubularly projecting structure extending beyond and recessing the nose, a back end portion with an opening, and a longitudinally extending wall between the nose and the back end portion;
an elongated plunger disposed in the back end portion in sliding sealed contact with the longitudinally extending wall of the housing, the plunger having a front end portion with a plunger seal and a back end portion carrying an end cap; and
a variable fluid chamber disposed in the housing between the nose and the front end portion of the plunger.

25. The syringe of claim 24 wherein the tubularly projecting structure is internally threaded.

26. The syringe of claim 25 wherein the tubularly projecting structure is sized to accept a flange from a luer lock needle attachment.
